(19) United States
(12) Patent Application Publication
(10) Pub. No.: US 2015/0173725 A1
(43) Pub. Date: Jun. 25, 2015

(54) Motion-Sensing Footswitch and Methods for a Surgical System

(71) Applicant: Alcon Research, Ltd., Fort Worth, TX (US)

(72) Inventors: Matthew Douglas Maxson, Tustin, CA (US); Ahmad Salehi, Irvine, CA (US)

(21) Appl. No.: 14/136,108

(22) Filed: Dec. 20, 2013

(51) Int. Cl. A61B 17/00

(52) U.S. Cl. CPC A61B 17/00; A61B 2017/00017; A61B 2017/00973

(57) ABSTRACT

A motion-sensing input device for control of a microsurgical system is disclosed, comprising a housing including a plurality of walls defining a cavity, a plurality of motion-sensing screens, and a processor. Each motion sensing screen of the plurality of motion-sensing screens is mounted within the cavity of the housing, and each motion-sensing screen is configured to detect positional data of an object within the cavity relative to the housing. The processor is operable to analyze the positional data and transmit corresponding command signals to the microsurgical system.

### BACKGROUND

Footswitches are an accepted part of the operator controls that enable the use of microsurgical and ophthalmic systems. This disclosure describes a footswitch in terms of its use with microsurgical systems and, in particular, its use with ophthalmic microsurgical systems.

When surgically treating a patient during ophthalmic surgery, a surgeon may use a complex patient treatment apparatus/surgical system that may require the control of a variety of different pneumatic and electronically driven subsystems. Typically, the operation of these subsystems is controlled by a microprocessor-driven console. The microprocessor within the surgical console may receive mechanical inputs from either the surgeon or from an assistant to the surgeon. During ophthalmic surgery, the surgeon views the patient's eye through an operating microscope while operating the system with both hands. To control the microsurgical system and its associated handpieces during the surgical procedure, the surgeon typically either instructs another healthcare professional how to alter the machine settings on the surgical system, or uses a footswitch to change the settings. When possible, many surgeons prefer to use the footswitch to alter the machine settings on the surgical system, eliminating or reducing the need to rely on another healthcare professional to adjust the system settings throughout the surgical procedure.

In some instances, an assistant may directly manipulate controls on the surgical console, while the surgeon/operator may use a control input device, such as a footswitch, to provide mechanical inputs. In the case of a footswitch, the mechanical inputs originate from the movement of the surgeon's foot to control the operation of a subsystem within the surgical system. The mechanical inputs are translated into electrical signals that are then fed to the microprocessor to control the operational characteristics of the desired subsystem. One example of such a subsystem is a laser system used in ophthalmic laser eye surgery, such as the EYELITE® photocoagulator manufactured by Alcon Laboratories, Inc. of Irvine, Calif.

The footswitch is capable of movement by the surgeon in a given range of motion to provide control of the functions of the surgical system or an associated handpiece. The footswitch may include a footpedal or tiltable treadle movably mounted to the base, similar to the accelerator pedal used to govern the speed of an automobile. The footpedal may be movable within set upward and downward limits. In one method of operation, the downward travel of the footpedal is resisted by different spring-like elements and different ranges of travel. The footpedal position is sensed and the operation of the surgical instrument is altered depending on which area of motion the footpedal is in. The movement of the footpedal typically provides a linear control input, and the range of motion of the footpedal may be segregated into several areas, each of which controls a different surgical mode or surgical function. Such linear control inputs may be used, for example, for regulating vacuum, rotational speed, power, or reciprocal motion. However, such mechanical footswitches may be limited in their ability to precisely translate the surgeon's movements into the desired surgical action.

Accordingly, there exists a need for an improved control input device for microsurgical systems, such as a surgical footswitch. The devices, systems, and methods disclosed herein overcome one or more of the deficiencies of the prior art.

### SUMMARY

This disclosure relates generally to a motion-sensing control input device for use during a surgical procedure, and in particular to a motion-sensing footswitch controlling an ophthalmic microsurgical system.

In an exemplary embodiment, a motion-sensing input device for control of a microsurgical system comprises a housing and a plurality of motion-sensing elements. In one aspect, the housing includes a plurality of walls defining a cavity, and each motion sensing screen of the plurality of motion-sensing elements is mounted within the cavity of the housing. In one aspect, each motion-sensing element is configured to detect positional data of an object within the cavity relative to the housing. In one aspect, the device includes a processor operable to analyze the positional data and transmit corresponding command signals to the microsurgical system.

In another exemplary embodiment, an ophthalmic microsurgical system comprises a handpiece and a motion-sensing footswitch. In one aspect, the handpiece has a plurality of functions. In one aspect, the motion-sensing footswitch comprises a housing defining a cavity and a motion-sensing element. In one aspect, the motion-sensing element is coupled to the housing. In one aspect, the motion-sensing element is configured to detect and track a position and an orientation of a foot movably positioned within the cavity and convey positional data representative of the detected position and orientation. In one aspect, the system includes a processor operable to receive the positional data and transmit corresponding command signals to the handpiece to selectively activate at least one of the plurality of functions based on the positional data.

In another exemplary embodiment, a method of controlling an ophthalmic microsurgical system by a motion-sensing footswitch comprises detecting a position and an orientation of an object within the motion-sensing footswitch with at least one motion-sensing element. In one aspect, the detected position and orientation correspond to surgical parameters controllable by the footswitch. In one aspect, the method includes transmitting the detected position and orientation of the object to a processor. In one aspect, the method includes generating a corresponding command signal based on the detected position and orientation of the object. In one aspect, the method includes relaying the corresponding command signal to an appropriate component of the ophthalmic microsurgical system.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 illustrates a perspective view of a microsurgical system including an exemplary motion-sensing footswitch according to one embodiment of the present disclosure.

FIG. 2 illustrates a perspective view of the exemplary motion-sensing footswitch shown in FIG. 1 according to one embodiment of the present disclosure.

FIG. 3 illustrates a side, cross-sectional view of the motion-sensing footswitch shown in FIG. 2 according to one embodiment of the present disclosure.

FIG. 4 illustrates a top, cross-sectional view of the motion-sensing footswitch shown in FIG. 2 according to one embodiment of the present disclosure.

FIG. 5 is a flow diagram illustrating an exemplary method of using an exemplary motion-sensing footswitch to control an exemplary surgical system according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure relates generally to motion-sensing devices used in the operation of microsurgical systems. In some instances, embodiments of the present disclosure are configured to be part of an ophthalmic surgical system. Instead of controlling aspects of the microsurgical system with the use of a mechanical footswitch, the devices disclosed herein allow the user to control the microsurgical system using a motion-sensing user interface or motion-sensing footswitch that recognizes and translates user motions into corresponding control signals for the microsurgical system. In some aspects, the devices, systems, and methods disclosed herein reduce or eliminate the need of a physical, mechanical footswitch and/or mechanical remote control to control the microsurgical system. By relying on the finely nuanced gestures of the user to control the microsurgical system, some aspects of the devices, systems, and methods disclosed herein may allow more precise control of microsurgical systems than conventional footswitches, thereby allowing for a greater degree of control over microsurgical procedures. In some embodiments, the devices, systems, and methods disclosed herein may utilize less power to control the microsurgical systems than conventional footswitches, thereby allowing for wirelessly operated, battery powered control devices.

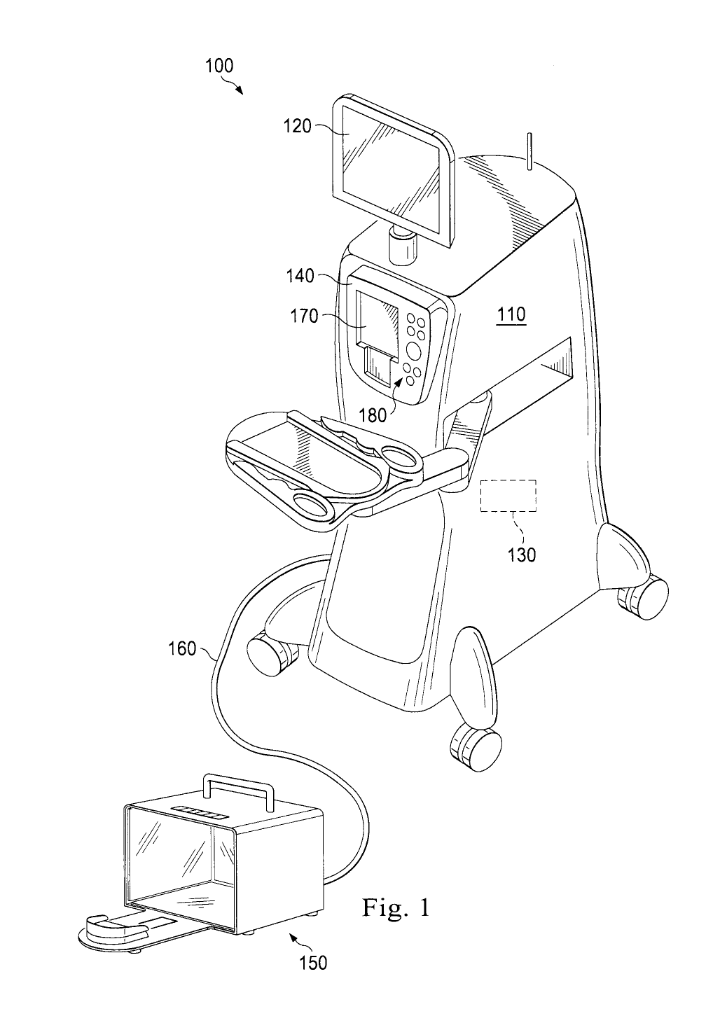

FIG. 1 illustrates a microsurgical system 100 according to one embodiment of the present disclosure. Though the microsurgical system 100 shown in FIG. 1 is an ophthalmic microsurgical system, the microsurgical system may be any microsurgical system, including a system for performing otic, nasal, throat, maxillofacial, or other surgeries. The system 100 is capable of providing ultrasound power, irrigation fluid, and aspiration vacuum to an ultrasonic handpiece in an anterior segment ophthalmic surgical procedure. The system 100 may also be capable of providing pneumatic drive pressure and aspiration vacuum to a vitrectomy probe and irrigation fluid to an irrigation cannula in a posterior segment ophthalmic surgical procedure.

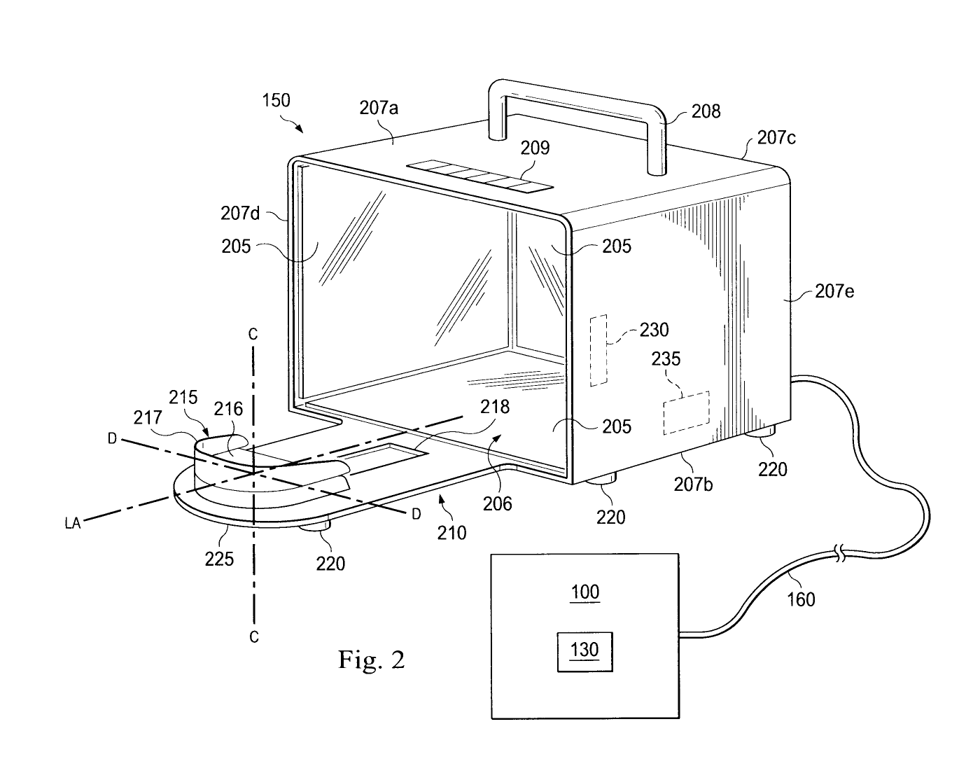

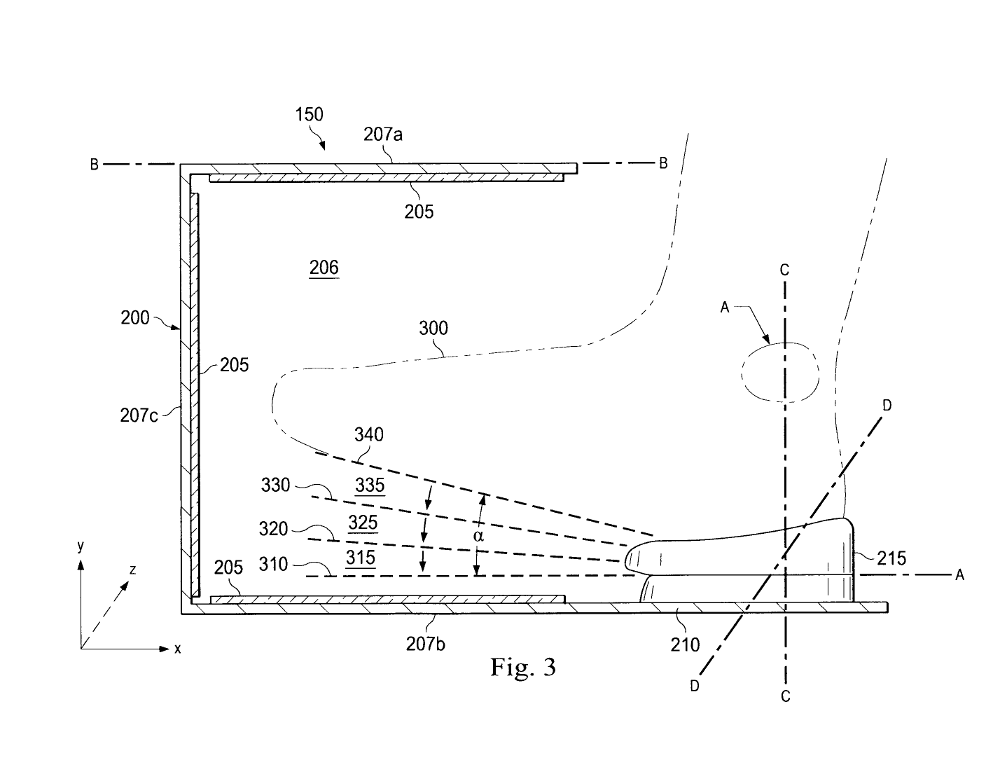

In the pictured embodiment, the system 100 includes a body 110, a graphical user interface (GUI) 120 attached to the body 110, a footswitch interface controller (FIC) 130 disposed within the body 110, a control console 140 disposed on a surface of the body 110, and a motion-sensing user interface or motion-sensing footswitch 150 connected to the FIC 130 via a bi-directional bus or cable 160. In some embodiments, the GUI 120 has a liquid crystal display (LCD) with touch screen capability. In other embodiments, the GUI 120 may include any of a variety of display devices, including by way of non-limiting example, Light Emitting Diode (LED) displays, Cathode Ray Tube (CRT) displays, and flat panel screens. The GUI 120 may include additional input devices or systems, including by way of non-limiting example, a keyboard, a mouse, a joystick, dials, buttons, among other input devices. The control console 140 includes a cassette receiving area 170 and a plurality of ports 180. A surgical cassette may be operatively coupled to the system 100 via the cassette receiving area 170 to manage the fluidics of the system 100 in a conventional manner. The bi-directional bus 160 sends signals in either direction between the FIC 130 and the motion-sensing footswitch 150, and may be used to transmit data to and from the motion-sensing footswitch 150 and/or may be used to transmit power to the motion-sensing footswitch 150. In some embodiments, the FIC 130 and the motion-sensing footswitch 150 communicate through a wireless connection. The FIC 130 is discussed in further detail below with reference to FIGS. 2-3.

During ophthalmic surgery, a series of handpieces may be coupled to the system 100, typically via conventional flexible plastic tubing fluidly coupled with the surgical cassette and/or electric cabling to operatively connect to the system 100 through one or more of the ports 180. Some exemplary handpieces that are utilized in anterior segment ophthalmic surgery include, for example, an irrigation handpiece, an irrigation/aspiration handpiece, an ultrasonic handpiece, and a diathermy handpiece. One type of exemplary ultrasonic handpiece is a phacoemulsification handpiece. Exemplary handpieces that are utilized in posterior segment ophthalmic surgery include, by way of non-limiting example, an extrusion handpiece, an infusion cannula, a vitrectomy probe, microsurgical scissors, and a diathermy handpiece.

The system 100 may include a microprocessor (within the FIC 130, the control console 140, and/or the motion-sensing footswitch 150), random access memory (RAM), read only memory (ROM), input/output circuitry such as the bus 160, an audio output device, and other components of microsurgical systems well known to those in the art. The memory may be used to store instructions executed by the processor as well as input/output data associated with the execution of the instructions. A variety of peripheral devices may also be coupled to the system 100, such as storage devices (hard disk drive, compact disc read-only memory (CD ROM) drive, etc.), printers, and other input/output devices.

FIG. 2 illustrates the motion-sensing footswitch 150 used to control various operational modes and functions of the microsurgical system 100 according to one embodiment of the present disclosure. The motion-sensing footswitch 150 includes a housing 200 that contains at least one motion-sensing element referenced herein as a motion-sensing screen 205. For example, in one embodiment, the housing 200 includes five motion-sensing screens 205. The housing 200 comprises a multi-sided, open structure configured to support the motion-sensing screens 205 within a cavity 206. In the pictured embodiment, the cavity 206 is a rectangular cavity defined by the walls 207. In the pictured embodiment, the housing 200 includes five walls 207: an upper wall 207a, a lower wall 207b, a front wall 207c, a left wall 207d, and a right wall 207e. In the pictured embodiment, a motion-sensing screen 205 is mounted on each wall 207. In other embodiments, the housing 200 may contain less motion-sensing screens 205 than walls 207. For example, in one embodiment, the lower wall 207b of the motion-sensing footswitch 150 may lack a motion-sensing screen. In other embodiments, the housing 200 may include multiple motion-sensing screens or other motion-sensing sensors on any single wall 207.

In the pictured embodiment, the housing 200 includes a handle 208 to enable a user to easily lift, reposition, and carry the motion-sensing footswitch 150. Alternative embodiments may lack a handle 208. In the pictured embodiment, the housing 200 include switches or buttons 209 that may be used by the surgeon to change various operating characteristics of the footswitch 150 and/or the system 100. Other embodiments may lack switches or buttons 209.

In the pictured embodiment, the motion-sensing footswitch 150 includes a foot plate 210 coupled to the housing 200. In some embodiments, the foot plate 210 is an integral part of the housing 200. In alternative embodiments, the foot plate 210 is a detachable part of the housing 200 or is entirely separate from the housing. The motion-sensing footswitch 150 includes a heel cup 215 that is shaped and configured to support and stabilize the heel of a user during use of the motion-sensing footswitch 150. The heel cup 215 secures the surgeon's heel relative to the motion-sensing screens 205 and guards against inadvertent slippage off the motion-sensing footswitch 150. The heel cup 215 is configured to allow the user to rest his or her heel on a platform 216 and against a sidewall 217 and extend the rest of his or her foot into the cavity 206 of the housing 200. In some embodiments, the platform 216 may be textured to provide frictional engagement with the surgeon's foot.

Unlike conventional footswitches comprising a movable footpedal to translate the user's motions in two dimensions (e.g., up and down along a vertical axis), the motion-sensing footswitch 150 lacks a physical support structure for the rest of the user's foot. Rather, the motion-sensing footswitch 150 is configured to allow the user to freely move his or her foot in at least three dimensions (e.g., right and left along a horizontal axis as well as up and down along a vertical axis). In the pictured embodiment, the platform 216 is raised above the level of the foot plate 210, thereby allowing the user to pivot his or her foot in a downward direction toward the lower wall 207b of the housing 200.

In the pictured embodiment, the heel cup 215 is positioned on the foot plate 210. Some embodiments lack the foot plate 210, and the heel cup 215 is coupled directly to the housing 200. For example, in some embodiments, the heel cup 215 is disposed within the housing 200. In some embodiments, the heel cup 215 may be an integral extension of either the foot plate 210 or the housing 200. In other embodiments, the heel cup 215 may be a separate component that is coupled to either the foot plate 210 or the housing 200by any of a variety of known methods, including by way of non-limiting example, adhesive, welding, and/or mechanical fasteners.

In some embodiments, the heel cup 215 may be repositionable relative to the front wall 207c on the motion-sensing footswitch 150 to increase or decrease the space available to accommodate for variations in the length of a user foot. For example, in the pictured embodiment, the heel cup 215 is slidable on a track 218 along a longitudinal axis LA of the foot plate 210. The heel cup 215 may be locked in position on the track 218 at a desired distance from the front wall 207c. In such an embodiment, the user may reposition and lock the heel cup 215 on the track 218 to optimally position his or her foot within the housing 200 for motion capture by the motion-sensing screens 205.

In some embodiments, the right wall 207e, the left wall 207d, the front wall 207c, and the upper wall 207a are in a fixed position relative to the lower wall 207b. In other embodiments, the right wall 207e, the left wall 207d, the front wall 207c, and/or the upper wall 207a may be adjusted inwardly or outwardly relative to the lower wall 207b to decrease or increase the space available between the walls 207 and accommodate for variations in the dimensions of a user foot. Thus, the motion-sensing footswitch may be ergonomically customized for different individual users.

The housing 200, the foot plate 210, and the heel cup 215 may be made from any suitable material or combination of materials, including, by way of non-limiting example, stainless steel, titanium, and/or plastic.

In the pictured embodiment, the housing 200 is attached to a plurality of base members 220 that support the motion-sensing footswitch 150 on the operating room floor. In other embodiments, the base member 210 comprises a single, continuous piece of material that covers the lower wall 207b of the housing 200 and a lower surface 225 of the foot plate 210 to support the motion-sensing footswitch 150 on the operating room floor. During operation, the motion-sensing footswitch 150 may be maintained in a constant position on the floor by the pressure of the user's heel pressing downward on the heel cup 215. In some embodiments, the base members 210 are configured to provide frictional resistance to the inadvertent sliding of the motion-sensing footswitch 150 across the operating room floor.

In the pictured embodiment, the motion-sensing screens 205 comprise flat-panel screens shaped and configured as continuous, substantially planar surfaces overlying the walls 207. In some embodiments, the motion-sensing screens 205 have at least some similar physical characteristics to conventional computer screens and/or touchscreens, and include motion-sensing capability. In the pictured embodiment, the motion-sensing footswitch 150 includes five motion-sensing screens 205 coupled to the housing to lie in parallel with their respective walls 207. In alternative embodiments, one or more of the motion-sensing screens 205 may be angled into the cavity 206 at nonparallel angles with their respective walls 207. Other embodiments may include any number of motion-sensing screens 205, positioned on any number of the walls 207 in any of a variety of arrangements (e.g., both symmetrical and nonsymmetrical, both fixed and repositionable). In addition, although described as screens, the motion-sensing screens 205 may be one or more discrete points, spots, or locations on the walls 207 of the housing 200.

The motion-sensing screens 205 may be any devices capable of motion capture having the ability to capture (e.g., identify and/or track) the movement of an object in three-dimensional (3D) space and translate that motion into another representation such as, by way of non-limiting example, a digital model. For example, the motion-sensing screens 205 may include any type or any number of cameras, including visible-light cameras, infrared (IR) cameras, ultraviolet cameras, or any other type of device or combination of devices that are capable of capturing an image of an object and representing that image in the form of digital data. In one example, each motion-sensing screen 205 comprises a grid of camera sensors, such as complementary metal-oxide-semiconductor (CMOS) active pixel sensors, and/or infrared (IR) light-emitting diodes (LEDs). In some embodiments, the motion-sensing screens 205 are able to capture the movement of objects with sub-millimeter precision within a 3D spatial volume (e.g., within the cavity 206 of the housing 200).

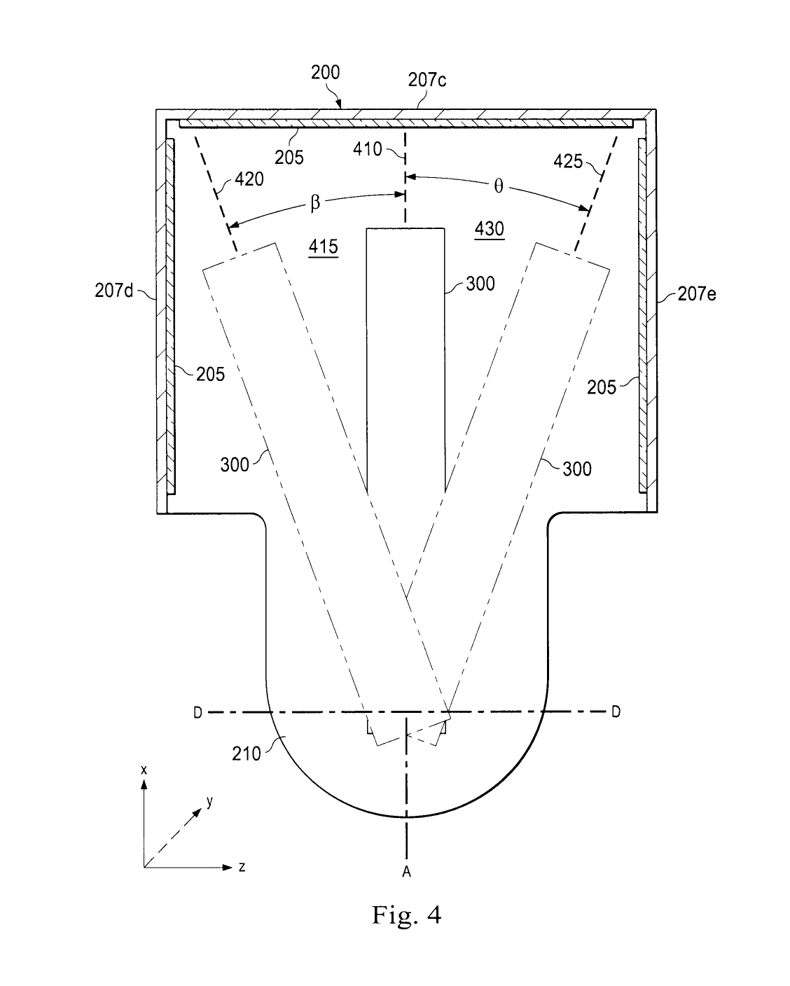

The motion-sensing screens 205 may be capable of capturing video images (i.e., successive image frames at a constant rate) to capture the motion of the object. The particular type and capabilities of the motion-sensing screens 205 can vary between different embodiments as to the frame rate, the image resolution, the color/intensity resolution, the imaging modality, the physical characteristics of the screens, depth of field, etc. In general, any imaging modality capable of focusing on and tracking an object within a spatial volume of interest may be used. In some instances, the motion-sensing screens 205 and the microsurgical system 100 includes components or features similar to those disclosed in U.S. Patent Publication No. 2013/0182897, entitled “Systems and Methods for Capturing Motion in Three-Dimensional Space,” and filed on Dec. 21, 2012, which is hereby incorporated by reference in its entirety. The motion-sensing screens 205 will be discussed in further detail below with reference to FIGS. 3 and 4.

In the pictured embodiment, the housing 200 includes a microprocessor 230 to allow for efficient communication with other system components, such as the motion-sensing screens 205 and/or the FIC 130. The microprocessor 230 is in communication with the motion-sensing screens 205 and the FIC 130 of the microsurgical system 100. The microprocessor 230 may be configured to translate and communicate the image data received by the motion-sensing screens 205 to the FIC 130. The microprocessor 230 may include one microprocessor chip, multiple processors and/or co-processor chips, and/or digital signal processor capability. In other embodiments, the body may lack the microprocessor 230 and therefore, processing and control may be entirely performed on the FIC 130 of the microsurgical system 100. In such embodiments, the motion-sensing screens 205 may be capable of data transformation and data transfer to and from the FIC 130 without the aid of a separate processor. The microprocessor 230 and/or the FIC 130 are operable to analyze the image data received from the motion-sensing screens 205 to determine the 3D position and motion of an object (e.g., a foot) that moves in the field of view of the motion-sensing screens 205 and translate that information into the appropriate control signals to control the microsurgical system 100.

In some embodiments, the motion-sensing screens 205 and/or the microprocessor 230 may be communicatively coupled to the FIC 130 via the cable 160. The cable 160 extends from and connects the motion-sensing footswitch 150 to the body 110 of the system 100 and provides electrical communication therebetween and provides power to the motion-sensing footswitch 150. In one embodiment, the motion-sensing footswitch 205 is a wireless footswitch and contains its own power source 235. The power source 235 may be a rechargeable battery, such as a lithium ion or lithium polymer battery, although other types of batteries may be employed. In addition, any other type of power cell is appropriate for power source 235.

In other embodiments, the motion-sensing screens 205 and/or the microprocessor 230 can communicate with and transfer data to the FIC 130 via wireless means. In wireless embodiments, communication between the FIC 130 and the microprocessor 230 or between the FIC 130 and the motion-sensing screens 205 may occur through a series of transmitting and receiving components onboard the motion-sensing footswitch 150 and within the body 110.

FIG. 3 illustrates a side, cross-sectional view of a foot 300 of the user (e.g., a surgeon) positioned within the motion-sensing footswitch 150 according to one embodiment of the present disclosure. FIG. 4 illustrates a top, cross-sectional view of the foot 300 positioned within the motion-sensing footswitch 150 according to one embodiment of the present disclosure.

In particular, in FIG. 3, the foot 300 is shown in an elevated position with the user resting a heel 305 on the platform 216 and against the sidewall 217 and extending the rest of the foot 300 into the cavity 206 of the housing 200. In the pictured embodiment, the foot 300 is shown elevated at an angle α relative to the platform 216 of the heel cup 215. When the foot 300 is lowered to align with a longitudinal axis A of the platform 216, the foot 300 is positioned in alignment with a neutral or resting plane represented by the longitudinal axis A. In some embodiments where the platform 216 is not elevated and is aligned with the foot plate 210, the axis A is substantially the same as the axis LA shown in FIG. 1. In the pictured embodiment, the foot 300 is movable about an axis C, which may extend through the user's ankle A and is substantially perpendicular to a longitudinal axis B of the upper wall 207a of the housing 200, and a longitudinal axis D extending through the heel cup 215. The foot is tiltable or pivotable with respect to the housing 200 about the axis D, which extends perpendicular to the axis C and parallel to the axis B in the pictured embodiment. The axes A, B, C, and D may be oriented differently relative to one another in other embodiments. In various embodiments, the axes A, B, C, and D may be oriented at known angles with respect to each other to enable effective and precise motion-sensing by the motion-sensing screens 205.

The user may move the foot 300 in a given range of motion (i e, limited by the dimensions of the housing 200, the capacities of the motion-sensing screens 205, and/or the flexibility of the foot 300) to change various operating characteristics of the microsurgical system 100. For example, the user can move the foot 300 in three dimensions to adjust the operational mode and/or provide proportional control to various operational functions of the microsurgical system 100. In some embodiments, as shown in FIG. 3, the user may move the foot 300 to rotate or pivot from about zero to about sixty degrees in the x-y plane about the axis D extending through the heel cup 215. In some embodiments, as shown in FIG. 4, the user may move the foot 300 to rotate or pivot from about zero to about 75 degrees about the axis C extending through the heel cup 215 (e.g., perpendicular to the axis D and the upper wall 207a). Other ranges of rotation are contemplated. These ranges of motion are typically segregated into several (virtual or digital) positions, each of which controls a different surgical mode. Neighboring positions frame or bookend distinct areas that provide proportional control over the functions of particular operational mode defined by each particular position. In some embodiments, movement in the x-y plane about the axis D controls both switching between different surgical modes and proportional control within those modes. In other embodiments, movement in the x-y plane about the axis D controls either switching between different surgical modes or proportional control within those modes, while movement in the x-z plane controls the other of switching between different surgical modes or proportional control within those modes. These control relationships are presented for exemplary purposes only, and other control relationships are contemplated.

As the foot 300 rotates about the axis D (e.g., up and down between the upper wall 207a and the lower wall 207b) and/or shifts or rotates (e.g., right and left between the left wall 207d and the right wall 207e) with respect to the axis C to progress from one position to another, the surgeon may actively change the operational mode or function as the motion-sensing screens 205 identifies and tracks the movement and either the microprocessor 230 and/or the FIC 130 process the image data into command signals to control the microsurgical system 100. Although FIG. 3 illustrates the foot 300 positioned on the heel cup 215 while it is moving within the housing 200, in some instances the motion-sensing screens 205 are operable to accurately identify and track the movements of the foot 300 while the user freely moves the foot 300 within the housing 200 without keeping his or her heel connected to the heel cup 215.

By way of nonlimiting example, depending on the operating mode of the system 100, the foot 300 may be moved within the housing 200 to provide focusing control over a microscope, proportional control, stepped control, or ON-OFF powering of vitrectomy probe cut rate, vitrectomy probe aspiration vacuum, ultrasound handpiece power, and/or ultrasound handpiece aspiration flow rate. In the example shown in FIG. 3, for an exemplary phacoemulsification handpiece operatively coupled to system 100, according to one embodiment of the present disclosure, keeping the foot in a first position 310 may provide no active surgical operations. Moving the foot 300 through a first area 315 may provide a fixed amount of irrigation flow to a handpiece. Moving the foot 300 into a second position 320 may provide fixed irrigation flow and activate control of aspiration flow into the handpiece. Moving the foot 300 through the second area 325 may provide fixed irrigation flow and proportional, linear control of aspiration flow. Moving the foot 300 into a third position 330 may activate control of ultrasound power to the handpiece. Moving the foot 300 through the third area 335 towards a fourth position 340 may provide fixed irrigation flow, proportional, linear control of aspiration flow, and proportional, linear control of ultrasound power to the handpiece.

In the example shown in FIG. 4, for an exemplary phacoemulsification handpiece operatively coupled to system 100, according to one embodiment of the present disclosure, keeping the foot 300 in a first position 410, which aligns with the axis A, may provide no active surgical operations or may provide irrigation flow to the handpiece. The user may swing the foot leftward in the x-z plane about the axis C shown in FIG. 3 by an angle 13 from the axis A to hover or pause in a second position 420. Moving the foot 300 through a first area 415 toward the left wall 207d and its corresponding motion-sensing screen 205 to the second position 420 may change the operational mode (e.g., into aspiration mode). In another instance, moving the foot 300 through a first area 415 to the second position 420 may provide a fixed amount of irrigation flow to the handpiece. The user may swing the foot rightward in the x-z plane about the axis C by an angle θ relative to the axis A to hover or pause in a second position 420. Moving the foot 300 into a third position 425 may activate control of ultrasound power to the handpiece. In another instance, moving the foot 300 into a third position 425 may provide fixed irrigation flow and activate control of aspiration flow into the handpiece. Moving the foot 300 through a second area 430 may provide fixed irrigation flow and proportional, linear control of aspiration flow.

In alternative embodiments, different numbers of positions and areas, either in the x-y plane or the x-z plane, as well as different surgical modes, may be assigned for different microsurgical systems other than system 100 and/or different handpieces operatively coupled to the system 100. In some embodiments, the number of positions and areas and the corresponding surgical modes may be defined by the surgeon using the control console 140 of the system 100. For example, the surgeon may customize the different positions and areas to particular surgical modes and proportional controls as desired. In one example, an upward motion of the foot 300 toward the upper wall 207a may be programmed into the system 100 as signifying an instruction to increase aspiration flow, whereas in another instance, the upward motion of the foot 300 may be programmed to signify an instruction to decrease aspiration flow.

In some embodiments, the surgeon can program the microsurgical system 100 (e.g., the microprocessor 230 and/or the FIC 130) to customize the control functions of particular movements. For example, the surgeon can customize whether, by way of non-limiting example: (1) movement in the x-y plane, as shown in FIG. 3, controls a footswitch-mediated transition through surgical modes while movement in the x-z plane, as shown in FIG. 4, controls a proportional shift in a particular action at the handpiece; (2) movement in the x-y plane, as shown in FIG. 3, controls a proportional shift in a particular action at the handpiece while movement in the x-z plane, as shown in FIG. 4, controls a footswitch-mediated transition through surgical modes; (3) movement in the x-y plane, as shown in FIG. 3, controls both proportional shift in a particular action at the handpiece and a footswitch-mediated transition through surgical modes; or (4) movement in the x-z plane, as shown in FIG. 4, controls both proportional shift in a particular action at the handpiece and a footswitch-mediated transition through surgical modes. Thus, two parameters (e.g., surgical mode, microscope control, and proportional control) could be controlled at one time by using motion in two planes. Moreover, the motion-sensing screens 205 offer limitless gesture-recognition capabilities to the user, and the user may assign specific motions or types of motions to particular command signals to increase the ease of use for the user.

The surgeon may freely move his or her foot 300 within the housing 200 to control the microsurgical system 100. Movement of the foot 300 within the housing 200, including the rotation of the foot 300 about the axes C and D, is detected by the motion-sensing screens 205 in three dimensions. As the foot 300 moves within the housing 200, the motion-sensing screens 205 identify and track the position of the foot 300 with respect to the walls 207 and/or the screens 205 as well as the rotational displacement of the foot 300 with respect to the axes C and D. In some instances, the motion-sensing screens 205 track the velocity of the movement and other characteristics of the motion. The rotational movement of the foot 300 with respect to the axes C and D may be precisely measured and cross-referenced by multiple motion-sensing screens 205 to achieve greater positional accuracy. For example, the positional and rotational measurements of individual motion-sensing screens 205 may be compared with each other to confirm and/or calculate accurate measurements.

The motion-sensing screens 205 detect the rotational displacement and position of the foot 300 and communicates data representative of the position of the foot 300 to the electronic components of the system 100 (e.g., the FIC 130) and/or motion-sensing footswitch 150 (e.g., the microprocessor 230). The motion-sensing screens 205 may communicate data corresponding to the sensed position of the foot 300 to the microprocessor 230 of the motion-sensing footswitch 150, or may include the circuitry necessary to communicate such data directly to the FIC 130 of the microsurgical system 100 (illustrated in FIGS. 1 and 2). The microprocessor 230 may report positional data to the FIC 130 of the microsurgical system 100 and/or may process command signals from the FIC 130 to control the motion-sensing screens 205. In alternative embodiments, the microprocessor 230 may independently issue command signals to control or adjust the motion-sensing screens 205 and/or surgical tools (e.g., the handpiece) of the microsurgical system 100. In alternative embodiments, the FIC 130 may independently issue command signals to control the motion-sensing screens 205 and/or surgical tool (e.g., the handpiece) of the microsurgical system 100.

The FIC 130 and/or the microprocessor 230 may include embedded software applications necessary to analyze the data from the motion-sensing screens 205 and to control the surgical tool (e.g., the handpiece) of the microsurgical system 100 based on that data. In particular, the software applications are designed to generate command signals based on the positional data received from the motion-sensing screens 205 to control the actuators of various components of the microsurgical system 100. Such software applications contain motion-sensing and control algorithms designed to read information from the control console 140 and/or motion-sensing screens 205 to create a distinct surgical effect for a given motion within the motion-sensing footswitch 150, whether the event is a footswitch-mediated transition through surgical modes or a proportional shift in a particular action at the handpiece. These software applications are designed to generate command signals based on the image data (including positional and rotational data) detected, sensed, or measured by the motion-sensing screens 205 to control the microsurgical system 100. In particular, the software application may use the data from the motion-sensing screens 205 to create command signals that can adjust the operating conditions and modes at the handpiece of the microsurgical system 100 without requiring the surgeon to shift his attention from the surgical field.

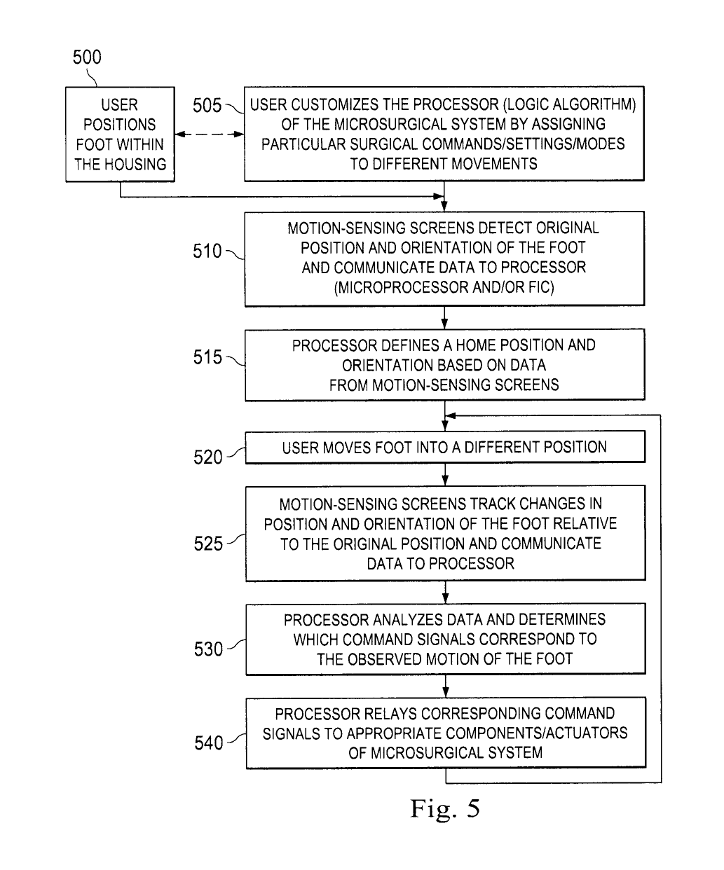

FIG. 5 is a flow diagram illustrating an exemplary method 500 of using the motion-sensing footswitch 150 to control the microsurgical system 100 according to one embodiment of the present disclosure. Initially, at 500, the surgeon positions his or her foot (e.g., foot 300) in a starting or original position within the housing 200 of the motion-sensing footswitch 150. In some embodiments, at 505, which may occur either before or after 500, the surgeon can customize the microsurgical system 100 (e.g., the microprocessor 230 and/or the FIC 130) by assigning particular surgical commands, parameters, settings, and/or modes to different movements and/or positions. In some embodiments, the surgeon may selectively activate and/or deactivate one or more motion-sensing screens 205.

At 510, the motion-sensing screens 205 detect the original position and orientation of the foot 300 relative to the housing 200, and communicate or transmit the data corresponding to the position and orientation of the foot 300 (i.e., positional data) to a processor such as the microprocessor 230 and/or the FIC 130. In some embodiments, the microprocessor 230 and/or the FIC 130 relays the positional data received from the motion-sensing screens 205 to the GUI 120, which displays the positional data and corresponding operational mode of the motion-sensing footswitch 150.

At 515, the microprocessor 230 and/or the FIC 130 define a home position and orientation based on the positional data detected by the motion-sensing screens 205.

At 520, the surgeon moves or rotates the foot 300 into a second position that is different than the first position.

At 525, the motion-sensing screens 205 track the changes in position and orientation of the foot 300 relative to the original or home position and communicate this positional data to the microprocessor 270 and/or the FIC 130.

At 530, the microprocessor 270 and/or the FIC 130 utilize embedded software applications to analyze the positional data received from the motion-sensing screens 205 to determine which command signals correspond to the observed motions of the foot 300. In some embodiments, a particular gesture may correspond to the command signal issued immediately previous to the recent repositioning of the foot 300. For instance, in one example, twitching the foot 300 to the left could correspond to the command signal of the previous surgical step.

At 540, the microprocessor 270 and/or the FIC 130 generate and relay command signals based on the positional data received from the motion-sensing screens 205 to control and/or actuate the appropriate components of the microsurgical system 100. In some embodiments, the microprocessor 230 contains the software applications necessary to control the microsurgical system 100 independently of the FIC 130. In such embodiments, the microprocessor 230 may issue command signals based on the data received from the motion-sensing screens 205 directly to the appropriate components of the microsurgical system 100. In other embodiments, the microprocessor 230 relays the positional data received from the motion-sensing screens 205 to the FIC 130 and receives command signals from the FIC 130, which contains the software applications necessary to generate the appropriate command signals. In some embodiments, the FIC 130 contains the software applications and circuitry necessary to receive the data from the motion-sensing screens 205 and control the microsurgical system 100 independently of the microprocessor 230. Such embodiments may lack a microprocessor 230.

In some instances, the motion-sensing devices, systems, and methods disclosed herein could be utilized by healthcare professionals other than the operating surgeon to change surgical parameters or power settings of the microsurgical system without being within close proximity of the GUI 120. For example, some embodiments may include motion-sensing screens 205 positioned outside the footswitch 150 (e.g., on the body 110 shown in FIG. 1). Persons who are not in close proximity to the microsurgical system 100 may gesture in view of the motion-sensing screens 205 to control the microsurgical system 100 from a distance.

The devices, systems, and methods disclosed herein may enable the motion-sensing footswitch to provide more precise control over the surgical parameters and power settings of the microsurgical system than a conventional mechanical footswitch. The devices, systems, and methods disclosed herein may allow the surgeon to freely move within more dimensions than a conventional footswitch and to customize the control inputs to enable more convenient, customizable, and rapid control over the microsurgical system. In some instances, the motion-sensing footswitch is capable of controlling the microsurgical system using less power than conventional mechanical footswitches. The devices, systems, and methods disclosed herein may allow for faster and more efficient control of the microsurgical system without as much time lag as conventional mechanical footswitches between user input at the footswitch and command signal output from the microsurgical system. The motion-sensing footswitches disclosed herein may avoid the disadvantages of mechanical degradation and malfunction sometimes associated with conventional mechanical footswitches.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, although the motion-sensing control input device is described in relation to a “footswitch,” it is understood that a user may insert and move any other object (e.g., a hand, or an inanimate object) within the motion-sensing footswitch instead of a foot to control the microsurgical system. It is understood that such variations may be made to the foregoing without departing from the scope of the present disclosure. Accordingly, it is appropriate that the appended claims be construed broadly and in a manner consistent with the present disclosure.

We claim:

1. A motion-sensing input device for control of a microsurgical system, comprising:
a housing including a plurality of walls defining a cavity;
a plurality of motion-sensing elements, each motion sensing screen of the plurality of motion-sensing elements mounted within the cavity of the housing, wherein each motion-sensing element is configured to detect positional data of an object within the cavity relative to the housing; and
a processor operable to analyze the positional data and transmit corresponding command signals to the microsurgical system.

2. The motion-sensing input device of claim 1, wherein each of the plurality of motion-sensing elements is mounted to one of the walls of the plurality of walls of the housing.

3. The motion-sensing device of claim 2, wherein at least one of the plurality of motion-sensing elements is mounted substantially parallel to at least one of the plurality of walls.

4. The motion-sensing input device of claim 1, further comprising a foot plate coupled to and extending from the housing.

5. The motion-sensing input device of claim 3, further comprising a heel cup coupled to the foot plate.

6. The motion-sensing input device of claim 5, wherein the heel cup is repositionable relative to the housing.

7. The motion-sensing input device of claim 1, further comprising a heel cup coupled to the housing.

8. The motion-sensing device of claim 1, wherein the cavity comprises a rectangular cavity defined by the plurality of walls.

9. The motion-sensing device of claim 1, wherein each of the plurality of motion-sensing elements is configured to convey the positional data to the processor.

10. An ophthalmic microsurgical system, comprising:
a handpiece having a plurality of functions;
a motion-sensing footswitch comprising:
a housing defining a cavity;
a motion-sensing element coupled to the housing and configured to detect and track a position and an orientation of a foot movably positioned within the cavity and convey positional data representative of the detected position and orientation; and
a processor operable to receive the positional data and transmit corresponding command signals to the handpiece to selectively activate at least one of the plurality of functions based on the positional data.

11. The ophthalmic microsurgical system of claim 10, wherein the motion-sensing footswitch further comprises a heel cup coupled to the housing.

12. The ophthalmic microsurgical system of claim 11, wherein the heel cup is repositionable relative to the motion-sensing element.

13. The ophthalmic microsurgical system of claim 10, wherein the processor comprises a microprocessor coupled to the motion-sensing footswitch.

14. The ophthalmic microsurgical system of claim 10, wherein the processor comprises a footswitch interface controller positioned apart from the motion-sensing footswitch.

15. The ophthalmic microsurgical system of claim 10, wherein the housing comprises a plurality of walls and at least one of the plurality of walls carries the motion-sensing element.

16. The ophthalmic microsurgical system of claim 10, wherein at least one of the plurality of walls is repositionable relative to the others of the plurality of walls to change the dimensions of the cavity.

17. A method of controlling an ophthalmic microsurgical system by a motion-sensing footswitch, comprising:
detecting a position and an orientation of an object within the motion-sensing footswitch with at least one motion-sensing element, the detected position and orientation corresponding to surgical parameters controllable by the footswitch;
transmitting the detected position and orientation of the object to a processor;
generating a corresponding command signal based on the detected position and orientation of the object; and
relaying the corresponding command signal to an appropriate component of the ophthalmic microsurgical system.

18. The method of claim 17, further comprising detecting an original position and an original orientation of the object within the motion-sensing footswitch with at least one motion-sensing element to establish a home position and home orientation.

19. The method of claim 17, wherein the motion-sensing footswitch includes a plurality of motion-sensing elements, and further comprising selectively activating at least one of the plurality of motion-sensing elements.

20. The method of claim 17, further including assigning a first command signal to a first position and orientation of the object and assigning a second command signal to a second position and orientation of the object, wherein the first command signal is different than the second command signal, and the first position and orientation is different than the second position and orientation.
